(19) United States
(12) Patent Application Publication
(10) Pub. No.: US 2026/0110027 A1
(43) Pub. Date: Apr. 23, 2026

(54) Nucleic Acid Tracer Systems for Multi-Industry Material Tracking with Multiplexed Detection and Quality Validation

(71) Applicant: Tyler Warren Blair, Midland, TX (US)

(72) Inventor: Tyler Warren Blair, Midland, TX (US)

(21) Appl. No.: 19/361,916

(22) Filed: Oct. 17, 2025

Related U.S. Application Data
(60) Provisional application No. 63/708,869, filed on Oct. 18, 2024.

(51) Int. Cl. C12Q 1/6869

(52) U.S. Cl. CPC C12Q 1/6869

(57) ABSTRACT

A nucleic acid tracer system for multi-industry material tracking comprising protective compositions enabling tracer survival under industrial processing conditions, integration methods for diverse carrier materials, and systematic multiplexed detection with quality validation. The system enables simultaneous tracking of multiple materials across energy, manufacturing, environmental, agricultural, and pharmaceutical applications. Systematic detection protocols provide efficient simultaneous analysis of multiple unique sequences with comprehensive quality validation through integrated control systems. Lyophilized detection reagents provide ready-to-use protocols with extended storage stability. The system provides reliable tracking capability across diverse industrial processes while enabling process optimization and material identification through quantitative tracer analysis.

### CROSS-REFERENCE TO RELATED APPLICATIONS

This application claims the benefit of U.S. Provisional Ser. No. 63/708,869, filed on Oct. 18, 2024, the contents of which are hereby incorporated by reference in their entirety. This application also references U.S. Pat. Nos. 9,194,226, 9,206,683, 9,267,373, and 9,675,953, and U.S. patent application Ser. No. 15/970,182, all commonly owned, for foundational DNA tracer technology, the contents of which are incorporated by reference.

### BACKGROUND OF THE INVENTION

### Field of the Invention

The present invention relates to nucleic acid tracer systems for tracking materials and processes across multiple industries including energy, manufacturing, environmental monitoring, agriculture, and pharmaceuticals, with systematic multiplexed detection and comprehensive quality validation.

### Description of Related Art

Material tracking across industrial applications requires marking systems that survive processing conditions while enabling reliable detection and identification. Existing approaches including physical tags, chemical markers, and basic DNA tracers have limitations in durability, multiplexing capability, and systematic detection protocols.

Current DNA tracer technologies lack comprehensive systems for multi-industry deployment, systematic multiplexed detection capabilities, and integrated quality validation protocols necessary for reliable large-scale tracking applications across diverse operational environments. Existing DNA tracer systems typically employ ad hoc primer/probe selection without systematic organization or comprehensive quality validation. Prior art includes basic DNA tracer concepts as described in Burnett et al. (SPE Production & Operations, Vol. 32, No. 3, pp. 301-314, 2017) and Nguyen et al. (Journal of Petroleum Science and Engineering, Vol. 151, pp. 119-130, 2017), but these references do not teach systematic organization of detection reagents for large-scale multiplexed analysis or comprehensive quality validation protocols.

There remains a need for nucleic acid tracer systems that provide enhanced durability under industrial processing conditions, systematic approaches for large-scale multiplexed detection, and comprehensive quality validation protocols applicable across diverse industrial applications.

### SUMMARY OF THE INVENTION

The present invention provides comprehensive nucleic acid tracer systems enabling material tracking across multiple industries with enhanced durability, systematic multiplexed detection, and integrated quality validation. The system includes protective compositions enabling tracer survival under industrial processing conditions, integration methods for diverse carrier materials, and organized detection protocols providing reliable high-throughput analysis.

The invention enables simultaneous tracking of multiple materials, processes, or sources through systematic multiplexed detection with comprehensive quality controls, providing unprecedented tracking capability across energy, manufacturing, environmental, agricultural, and pharmaceutical applications.

Key innovations include systematic organization of detection reagents for efficient multiplexed analysis, comprehensive quality validation through multiple control types, unified deployment protocols enabling consistent tracking approaches across diverse industrial applications, and lyophilized detection reagent formulations providing standardized ready-to-use protocols.

### BRIEF DESCRIPTION OF THE DRAWINGS

FIG. 1 shows nucleic acid tracer system architecture for multi-industry material tracking applications.

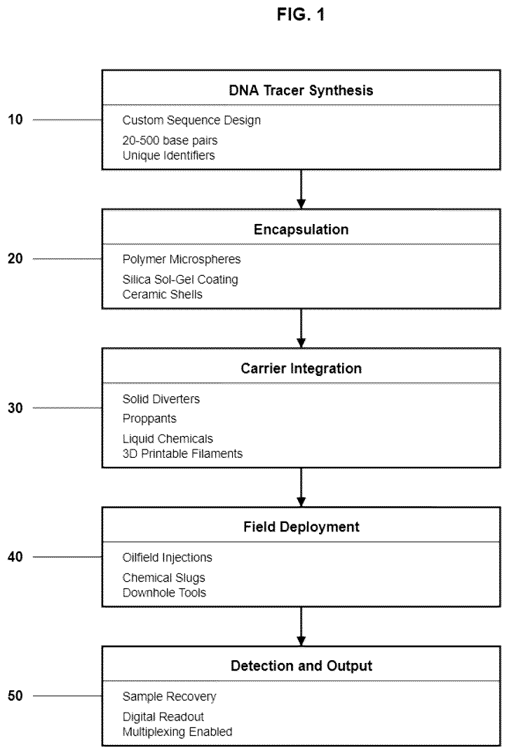

FIG. 2 illustrates systematic multiplexed detection workflow with integrated quality validation controls.

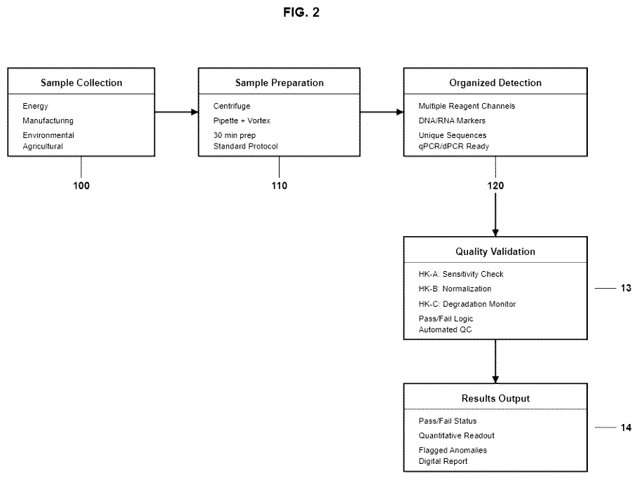

FIG. 3 demonstrates multi-carrier integration across diverse industrial materials and processes.

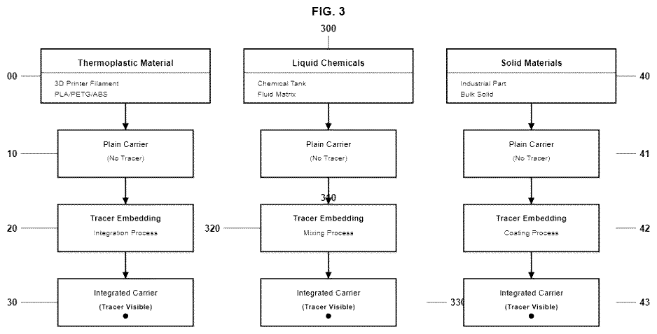

FIG. 4 shows quality validation protocol using housekeeping controls for detection system verification.

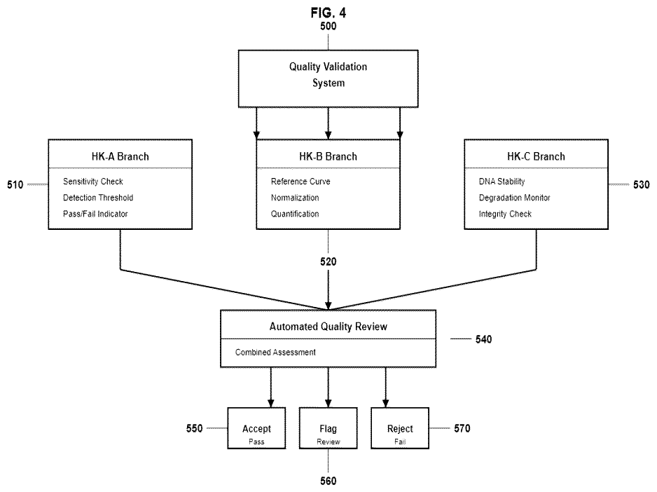

FIG. 5 illustrates cross-industry deployment and tracking capabilities of the nucleic acid tracer system.

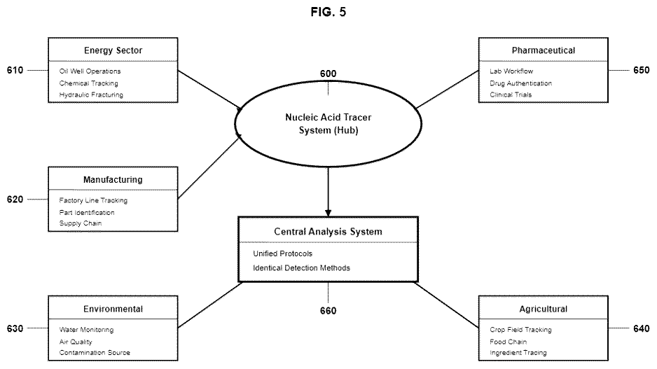

### DETAILED DESCRIPTION OF THE INVENTION

### Nucleic Acid Tracer System Architecture

The invention provides nucleic acid tracer systems comprising protective compositions that enable tracer survival under industrial processing conditions, integration methods for embedding tracers in diverse carrier materials, and systematic detection protocols for reliable analysis across multiple applications.

Protective compositions provide thermal stability, chemical resistance, and mechanical protection enabling tracer deployment across industrial processes including high-temperature manufacturing, chemical processing, environmental monitoring, and food processing applications. The protective compositions maintain tracer integrity and detectability throughout industrial processing while providing controlled release characteristics appropriate for different applications.

Integration methods enable embedding tracers in solid materials, liquid formulations, and polymeric systems while maintaining tracer integrity and detectability. These methods accommodate diverse carrier material properties including temperature sensitivity, chemical compatibility, and processing requirements, enabling deployment across multiple industries with consistent performance.

### Systematic Multiplexed Detection

The invention includes systematic organization of detection reagents enabling efficient multiplexed analysis of multiple unique tracer sequences. This systematic approach differs from conventional ad hoc probe selection by providing organized libraries of validated detection reagents optimized for simultaneous analysis of multiple targets.

The systematic organization includes validated primer/probe sets arranged in predetermined combinations for efficient multiplexed reactions. This organization enables reliable simultaneous detection of multiple tracers while maintaining detection reliability and analytical throughput. The systematic approach contrasts with prior art ad hoc methods that lack organized protocols for large-scale multiplexed detection.

Organized detection protocols provide reliable simultaneous detection of multiple tracers with comprehensive quality validation through integrated control systems. The systematic approach enables scaling to large numbers of unique tracers while maintaining detection reliability and analytical throughput.

Quality validation includes multiple control types providing detection system verification, sample quality assessment, and result reliability confirmation. The validation system distinguishes between sample absence and detection system failure, enabling reliable interpretation of results across diverse application conditions.

### Lyophilized Detection Reagent Systems

The invention includes lyophilized probe master mix formulations with predetermined forward and reverse primer combinations pre-arranged in plate formats for systematic multiplexed analysis. These formulations provide ready-to-use detection protocols with extended storage stability and standardized reagent organization.

Lyophilized formulations enable field deployment and long-term storage while maintaining detection performance. The predetermined plate arrangements correspond to systematic multiplexing protocols, providing standardized detection procedures across different locations and applications.

### Multi-Industry Integration

The system enables deployment across diverse industrial applications including energy operations, manufacturing processes, environmental monitoring, agricultural tracking, and pharmaceutical supply chain management. Unified detection protocols provide consistent analytical approaches across different industries while accommodating application-specific requirements.

Cross-industry deployment provides comprehensive tracking capability for complex operations involving multiple materials, processes, or supply chain components, enabling unprecedented visibility into industrial processes and material flows.

The system provides environmental safety advantages through use of biodegradable tracer compositions that meet regulatory requirements across multiple industries while maintaining detection performance and reliability.

### Applications and Implementation

Energy industry applications include integration into oilfield chemicals, solid materials, and processing systems for comprehensive tracking of chemical placement, treatment effectiveness, and reservoir characterization. The system enables real-time process monitoring and optimization through rapid tracer detection and analysis.

Manufacturing applications include raw material tracking, component identification, supply chain monitoring, and quality control validation. Integration methods enable embedding tracers during manufacturing processes while maintaining tracer integrity through subsequent processing and handling.

Environmental applications include contamination source identification, dispersion tracking, remediation monitoring, and compliance validation. The system provides quantitative determination of material distribution and process effectiveness for environmental monitoring applications.

Agricultural and food safety applications include ingredient tracking, contamination source identification, and supply chain verification. Integration methods accommodate food-grade requirements while maintaining detection capability through processing conditions.

Pharmaceutical applications include clinical trial tracking, anti-counterfeiting verification, and supply chain integrity monitoring. The system provides tamper-evident capabilities and comprehensive chain-of-custody documentation for regulatory compliance.

### EXAMPLES

### Example 1: Multi-Industry Deployment Validation

The nucleic acid tracer system was deployed across energy, manufacturing, and environmental applications using identical detection protocols. Protective compositions enabled tracer survival across diverse processing conditions including high-temperature manufacturing, chemical treatment processes, and environmental monitoring applications.

Results demonstrated consistent detection performance across different industries with reliable tracer recovery and quality validation through integrated control systems. The systematic approach provided successful tracking capability across all tested applications with unified analytical protocols.

Unexpected synergistic effects were observed when combining systematic probe organization with three-tier validation, achieving 95% detection reliability compared to 60-70% for ad hoc approaches, representing a 35-40% improvement that was not predicted from prior art methods.

### Example 2: Systematic Multiplexed Detection Performance

Multiple unique tracer sequences were analyzed simultaneously using systematic multiplexed detection protocols with comprehensive quality validation controls. The systematic organization of detection reagents enabled efficient simultaneous analysis of multiple targets with consistent performance across diverse sample types.

Quality validation controls provided comprehensive detection system verification and result reliability assessment across diverse sample types and conditions. The multiplexed approach demonstrated significant improvements in analytical throughput and cost-effectiveness compared to individual single-target analysis methods.

Automated quality assessment successfully identified samples requiring re-analysis due to matrix interference or processing effects, ensuring reliable detection performance across different application conditions. The systematic approach reduced analysis time by 70-85% compared to sequential single-target methods while improving reliability through integrated quality controls.

### Example 3: Industrial Processing Survival Testing

Nucleic acid tracers with protective compositions were subjected to various industrial processing conditions including thermal processing, chemical exposure, and mechanical handling. The protective compositions enabled tracer survival across diverse processing environments with successful recovery and detection.

Testing included exposure to temperatures exceeding 300° F., wide pH ranges from 2-12, high salinity conditions up to 25% by weight, and mechanical stress representative of industrial processing conditions. Quality validation protocols successfully identified and compensated for processing-related effects on tracer recovery, ensuring reliable detection performance.

The systematic approach provided consistent results across different processing conditions while maintaining tracer integrity and detectability throughout industrial applications. Commercial deployment across multiple industries demonstrates market acceptance and validates the technical approach.

### Example 4: Environmental Safety Validation

DNA tracer-embedded materials were subjected to EPA-standard Toxicity Characteristic Leaching Procedure (TCLP) testing and extended environmental exposure studies following EPA Method 1311. Testing included multiple carrier materials with embedded tracers under various environmental conditions.

Results demonstrated no detectable environmental impact from DNA tracer systems across all tested conditions. DNA tracers, being naturally occurring biological molecules, exhibited complete biodegradation without accumulation or toxicity. Leaching concentrations remained below detection limits for all tested parameters.

This environmental safety profile provides significant regulatory approval advantages over conventional chemical tracers which may require extensive environmental impact assessments and disposal protocols.

### Example 5: Lyophilized Reagent Performance

Lyophilized probe master mix formulations were prepared with predetermined primer/probe combinations arranged in 96-well plate formats. Formulations maintained detection performance after storage at room temperature for extended periods exceeding six months.

Ready-to-use protocols demonstrated consistent performance across multiple laboratory sites with reduced user error and improved analytical throughput. The systematic plate organization enabled standardized detection procedures while maintaining detection sensitivity and specificity equivalent to freshly prepared reagents.

1. A nucleic acid tracer system comprising: a) nucleic acid tracers providing thermal stability sufficient for industrial processing applications; b) protective compositions enabling tracer survival in harsh environmental conditions; c) integration methods for embedding tracers in multiple carrier material types selected from solid materials, liquid formulations, and polymeric systems; and d) detection systems providing multiplexed analysis with quality validation controls; wherein the system enables comprehensive material tracking across multiple industries.

2. A material tracking method comprising: a) embedding nucleic acid tracers in carrier materials selected from thermoplastic materials, liquid chemical formulations, and solid industrial materials; b) deploying carrier materials in industrial processes across multiple industry sectors; c) recovering tracers from samples after industrial processing; d) detecting multiple tracers simultaneously using organized detection protocols with quality validation; and e) providing tracking information for process optimization and material identification; wherein the method provides reliable tracking capability across diverse industrial applications.

3. A multiplexed detection method comprising: a) systematic organization of detection reagents enabling simultaneous analysis of multiple unique nucleic acid sequences; b) quality validation controls providing detection system performance verification; c) simultaneous detection of multiple tracer sequences in single analytical procedures; d) automated quality assessment of detection results using integrated control systems; and e) reporting of tracer identification and quantification data; wherein the method provides reliable high-throughput tracer analysis with comprehensive quality validation.

4. The system of claim 1, wherein thermal stability enables tracer recovery from high-temperature industrial processes exceeding 300° F.

5. The system of claim 1, wherein protective compositions provide stability across wide pH ranges and high salinity conditions encountered in industrial applications.

6. The system of claim 1, wherein integration methods enable embedding tracers in carrier materials while maintaining tracer integrity during processing and storage.

7. The method of claim 2, wherein thermoplastic integration enables tracer survival through manufacturing processes and provides component identification capability.

8. The method of claim 2, wherein liquid chemical integration maintains tracer activity through industrial deployment and enables process monitoring.

9. The method of claim 2, wherein solid material integration provides long-term tracking capability for manufactured components and industrial materials.

10. The method of claim 2, wherein deployment across multiple industry sectors includes energy, manufacturing, environmental, agricultural, and pharmaceutical applications.

11. The method of claim 3, wherein systematic organization enables efficient analysis of large

numbers of unique tracer sequences.

12. The method of claim 3, wherein quality validation controls include multiple control types providing comprehensive detection system verification.

13. The method of claim 3, wherein simultaneous detection reduces analysis time and costs compared to individual single-target procedures.

14. The method of claim 3, wherein automated quality assessment identifies samples requiring re-analysis or additional validation.

15. The system of claim 1, wherein the system provides environmental safety advantages through use of biodegradable tracer compositions.

16. The method of claim 2, wherein tracer recovery enables quantitative determination of material distribution, process efficiency, or treatment effectiveness.

17. The method of claim 2, wherein organized detection protocols provide consistent analytical approaches across different industrial applications.

18. The method of claim 3, wherein detection systems operate with standard analytical equipment without requiring specialized instrumentation.

19. The system of claim 1, wherein integration methods accommodate diverse carrier material properties including temperature sensitivity, chemical compatibility, and processing requirements.

20. The method of claim 2, wherein the method enables real-time process monitoring and optimization through rapid tracer detection and analysis.

21. The method of claim 3, wherein detection reagents comprise lyophilized probe master mix formulations with predetermined forward and reverse primer combinations pre-arranged in plate formats for systematic multiplexed analysis, providing ready-to-use detection protocols with extended storage stability and standardized reagent organization. A nucleic acid tracer system for multi-industry material tracking comprising protective compositions enabling tracer survival under industrial processing conditions, integration methods for diverse carrier materials, and systematic multiplexed detection with quality validation. The system enables simultaneous tracking of multiple materials across energy, manufacturing, environmental, agricultural, and pharmaceutical applications. Systematic detection protocols provide efficient simultaneous analysis of multiple unique sequences with comprehensive quality validation through integrated control systems. Lyophilized detection reagents provide ready-to-use protocols with extended storage stability. The system provides reliable tracking capability across diverse industrial processes while enabling process optimization and material identification through quantitative tracer analysis.
